(19) United States
(12) Patent Application Publication
(10) Pub. No.: US 2009/0234394 A1
(43) Pub. Date: Sep. 17, 2009

(54) UNILATERAL FACET BOLT INSERTER

(76) Inventor: DAVID CROOK, Mineola, TX (US)

Correspondence Address: Clements Bernard PLLC, 1901 Roxborough Road, Suite 300, Charlotte, NC, 28211, US

(21) Appl. No.: 12/400,338

(22) Filed: Mar. 9, 2009

Related U.S. Application Data
(60) Provisional application No. 61/035,402, filed on Mar. 11, 2008.

(51) Int. Cl. A61F 5/00; A61B 17/88

(52) U.S. Cl. 606/86 A; 606/279

(57) ABSTRACT

The present invention provides a surgical device for securing two bony structures together, including: a handle; an elongated stem coupled to the handle; a housing coupled to the elongated stem; a bolt driving assembly disposed at least partially within the housing, wherein the bolt driving assembly is operable for selectively retaining a bolt and driving it through the two bony structures; and a bridge member coupled to the elongated stem, wherein the bridge member is selectively extensible from/retractable to the elongated stem and is operable for selectively retaining a first washer that is configured and positioned to selectively receive the bolt after it is driven through the two bony structures. The surgical device also includes a first actuation mechanism coupled to the elongated stem, wherein the first actuation mechanism is operable for actuating the bolt driving assembly. The surgical device further includes a second actuation mechanism coupled to the elongated stem, wherein the second actuation mechanism is operable for actuating the bridge member. One or more of the housing and the bolt driving assembly are operable for selectively retaining a second washer that is configured and positioned to selectively receive the bolt before it is driven through the two bony structures.

### CROSS-REFERENCE TO RELATED APPLICATION(S)

The present non-provisional patent application/patent claims the benefit of priority of U.S. Provisional Patent Application No. 61/035,402, filed on Mar. 11, 2008, and entitled “UNILATERAL FACET BOLT INSERTER,” the contents of which are incorporated in full by reference herein.

### FIELD OF THE INVENTION

The present invention relates generally to surgical devices and methods for performing bone arthrodesis. More specifically, the present invention relates to a unilateral facet bolt inserter.

### BACKGROUND OF THE INVENTION

Bone arthrodesis, or fusion, is a surgical procedure that is used to stabilize or immobilize impaired bones or joints such that they or other associated structures may heal. More specifically, facet arthrodesis is a surgical procedure that is used to stabilize or immobilize a spinal facet joint in the treatment of an injury or degenerative condition. Conventional facet arthrodesis systems and methods utilize bone screws that are driven through the superior and inferior facets so as to allow the adjoined bone sections or other associated structures to fuse together. Conventional facet arthrodesis systems and methods also utilize wires that are looped around the superior and inferior facets so as to allow the adjoined bone sections or other associated structures to fuse together. The surgical procedures that must be employed to implant these bone screws or wires are difficult and time consuming. Therefore, there is a need for improved surgical devices and methods for performing bone arthrodesis.

U.S. patent application Ser. No. 10/683,076 (U.S. Patent Application Publication No. 2004/0143268) (Falahee), Ser. No. 10/973,524 (U.S. Patent Application Publication No. 2005/0234459) (Falahee et al.), Ser. No. 12/122,498 (U.S. Patent Application Publication No. 2009/0054903) (Falahee et al.), and Ser. No. 12/388,959 (not yet published) (Cox) disclose conventional systems for performing bone arthrodesis that each include an implant for bone arthrodesis and a bone fastening device. The implant includes a fastener with an elongated shaft having a head at one end and a bone piercing point at the opposite end. A first washer has a structure for engaging the head of the shaft so as to be polyaxially pivotable with respect to the head. A locking member has a structure for engaging the shaft. The locking member may have a second washer pivotally engaged thereto. The bone fastening device may include a straight or curved elongated cannula with a collet for detachably engaging the first washer and for advancing the first washer. A structure is provided for engaging the fastener and for advancing and rotating the fastener through the collet and through the first washer. The bone arthrodesis device further includes a lower end portion extending from the cannula. The lower end portion has structure for detachably engaging the locking member. The fastener, first washer, and locking member are aligned such that the advancing fastener advances through the first washer, drills through the bone, and moves into the locking member. Methods for performing bone arthrodesis are also disclosed. Although an improvement, these facet arthrodesis systems are not ideal for all spinal morphologies or in all surgical applications. Therefore, there is still a need for improved surgical devices and methods for performing bone arthrodesis.

### BRIEF SUMMARY OF THE INVENTION

In one exemplary embodiment, the present invention provides a surgical device for securing two bony structures together, including: a handle; an elongated stem coupled to the handle; a housing coupled to the elongated stem; a bolt driving assembly disposed at least partially within the housing, wherein the bolt driving assembly is operable for selectively retaining a bolt and driving it through the two bony structures; and a bridge member coupled to the elongated stem, wherein the bridge member is selectively extensible from/retractable to the elongated stem and is operable for selectively retaining a first washer that is configured and positioned to selectively receive the bolt after it is driven through the two bony structures. Optionally, the housing is articulatable with respect to the elongated stem. The surgical device also includes a first actuation mechanism coupled to the elongated stem, wherein the first actuation mechanism is operable for actuating the bolt driving assembly. Optionally, the first actuation mechanism is operable for imparting rotational motion to the bolt driving assembly, whereby the bolt is rotated and translated through the two bony structures. The surgical device further includes a second actuation mechanism coupled to the elongated stem, wherein the second actuation mechanism is operable for actuating the bridge member. Optionally, the second actuation mechanism is operable for imparting rotational motion to the bridge member, whereby the bridge member is translated to the two bony structures. One or more of the housing and the bolt driving assembly are operable for selectively retaining a second washer that is configured and positioned to selectively receive the bolt before it is driven through the two bony structures. Optionally, the two bony structures are the superior and inferior facets of a facet joint of a spine of a patient.

In another exemplary embodiment, the present invention provides a surgical method for securing two bony structures together, including: providing a handle; providing an elongated stem coupled to the handle; providing a housing coupled to the elongated stem; providing a bolt driving assembly disposed at least partially within the housing, wherein the bolt driving assembly is operable for selectively retaining a bolt and driving it through the two bony structures; and providing a bridge member coupled to the elongated stem, wherein the bridge member is selectively extensible from/retractable to the elongated stem and is operable for selectively retaining a first washer that is configured and positioned to selectively receive the bolt after it is driven through the two bony structures. Optionally, the housing is articulatable with respect to the elongated stem. The surgical method also includes providing a first actuation mechanism coupled to the elongated stem, wherein the first actuation mechanism is operable for actuating the bolt driving assembly. Optionally, the first actuation mechanism is operable for imparting rotational motion to the bolt driving assembly, whereby the bolt is rotated and translated through the two bony structures. The surgical method further includes providing a second actuation mechanism coupled to the elongated stem, wherein the second actuation mechanism is operable for actuating the bridge member. Optionally, the second actuation mechanism is operable for imparting rotational motion to the bridge member, whereby the bridge member is translated to the two bony structures. One or more of the housing and the bolt driving assembly are operable for selectively retaining a second washer that is configured and positioned to selectively receive the bolt before it is driven through the two bony structures. Optionally, the two bony structures are the superior and inferior facets of a facet joint of a spine of a patient.

In a further exemplary embodiment, the present invention provides a surgical method for securing two bony structures together, including: disposing a bolt driving assembly and a bridge member on opposing sides of a joint comprising the two bony structures, wherein the bolt driving assembly is operable for selectively retaining a bolt and driving it through the two bony structures, and wherein the bridge member is selectively extensible from/retractable to the joint and is operable for selectively retaining a first washer that is configured and positioned to selectively receive the bolt after it is driven through the two bony structures; securing the two bony structures between the bolt driving assembly and the bridge member; and driving the bolt through the two bony structures and the first washer. Optionally, the bolt driving assembly is operable for selectively retaining a second washer that is configured and positioned to selectively receive the bolt before it is driven through the two bony structures. Optionally, the two bony structures are the superior and inferior facets of a facet joint of a spine of a patient.

### BRIEF DESCRIPTION OF THE DRAWINGS

The present invention is illustrated and described herein with reference to the various drawings, in which like reference numbers are used to denote like device components/method steps, as appropriate, and in which:

FIG. 1 is a side planar view of the unilateral facet bolt inserter of the present invention;

FIG. 2 is a perspective view of the unilateral facet bolt inserter of FIG. 1; and

FIG. 3 is a partial cross-sectional view of the unilateral facet bolt inserter of FIGS. 1 and 2.

### DETAILED DESCRIPTION OF THE INVENTION

In various exemplary embodiments, the present invention provides a unilateral facet bolt inserter. As described above, U.S. patent application Ser. No. 10/683,076 (U.S. Patent Application Publication No. 2004/0143268) (Falahee), Ser. No. 10/973,524 (U.S. Patent Application Publication No. 2005/0234459) (Falahee et al.), Ser. No. 12/122,498 (not yet published) (Falahee et al.), and Ser. No. 12/388,959 (not yet published) (Cox) disclose conventional systems for performing bone arthrodesis that each include an implant for bone arthrodesis and a bone fastening device. Advantageously, the unilateral facet bolt inserter of the present invention is better suited for various spinal morphologies and in some surgical applications. This unilateral facet bolt inserter is used to insert a facet bolt into a spinal facet joint for spinal fixation. The use of such facet bolts is a less invasive alternative to the use of pedicle screws and rods as a mechanism for creating a posterior tension band to support an interbody fusion, for example.

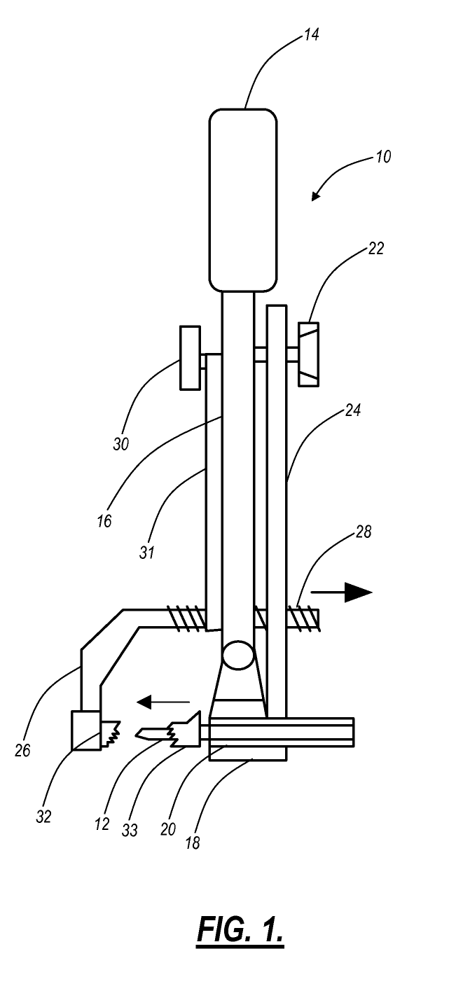

Referring to FIG. 1, in one exemplary embodiment, the unilateral facet bolt inserter 10 of the present invention is configured to insert a facet bolt 12 into a spinal facet joint (i.e. through the superior and inferior facets of the spinal facet joint) for spinal fixation. In general, the unilateral facet bolt inserter 10 includes a handle 14 connected to an elongated stem 16. In operation, a surgeon holds and manipulates the handle 14, which may be ergonomically designed, to position, secure, and actuate the unilateral facet bolt inserter 10, and thereby deploy the facet bolt 12 into the spinal facet joint for spinal fixation. Accordingly, the elongated stem 16 is placed in and through an incision and/or other access portal, such that the spinal facet joint may be visualized and accessed. Such access portals are well known to those of ordinary skill in the art. It should also be noted that the unilateral facet bolt inserter 10 may be used via any suitable conventional surgical approach (i.e. from any suitable angle with respect to the body of the patient).

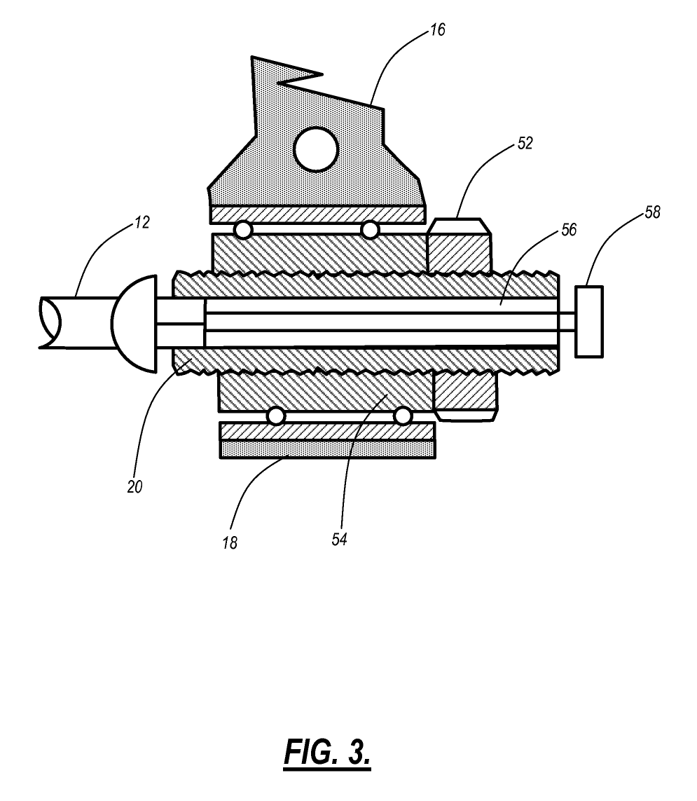

The elongated stem 16 extends to and terminates in a housing 18 (described in greater detail in FIG. 3). Optionally, the housing 18 is articulatable with respect to the elongated stem 16. The housing 18 includes an internally threaded mechanism 20 that is configured to selectively drive the facet bolt 12 into and through the spinal facet joint, preferably but not necessarily by imparting a rotational force to the facet bolt 12. A first actuation mechanism 22 is located on the elongated stem 16 for this purpose. In this exemplary embodiment, the first actuation mechanism 22 includes a knob that is rotatably connected to the elongated stem 16. A belt/chain drive system 24 or the like is rotatably connected to the first actuation mechanism 22, as well as to a gear 52 (FIG. 3) associated with the housing 18 and internally threaded mechanism 20. In operation, the surgeon rotates or otherwise actuates the first actuation mechanism 22, which imparts rotational force to or otherwise actuates the belt/chain drive system 24 or the like, which imparts rotational force to or otherwise drives the gear 52, thereby driving the facet bolt 12 into and through the spinal facet joint. Alternatively, a powered mechanism (not illustrated) may be attached to and actuate the belt/chain drive system 24 or the like responsive to the surgeon's command. The belt/chain drive system 24 or the like may be either substantially disposed within or external to the elongated stem 16 and, in the later case, it may be disposed within a separate housing (not illustrated).

Prior to driving the facet bolt 12 into and through the spinal facet joint, the unilateral facet bolt inserter 10 is positioned with respect to and secured to the spinal facet joint. This is accomplished using a bridge member 26 that is movably connected to the elongated stem 16 via an extensible threaded mechanism 28. The bridge member 26 is configured to receive the end of the facet bolt 12 after insertion through the spinal facet joint. Accordingly, either one or both of the housing 18 and the bridge member 26 may selectively retain a washer 32 and 33 through which the facet bolt 12 selectively passes, thereby retaining the facet bolt 12 in the spinal facet joint when the unilateral facet bolt inserter 10 is removed. One or both of these washers 32 and 33 may have any suitable bone engaging structures, such as friction surfaces, teeth, etc.

The bridge member 26 is configured to selectively move towards the facet bolt 12 prior to or during insertion to lock the unilateral facet bolt inserter 10 onto the spinal facet joint. A second actuation mechanism 30 is located on the elongated stem 16 for this purpose. In this exemplary embodiment, the second actuation mechanism 30 includes a knob that is rotatably connected to the elongated stem 16. A belt/chain drive system 31 or the like is rotatably connected to the second actuation mechanism 30, as well as to a gear (not illustrated) associated with the bridge member 26. In operation, the surgeon rotates or otherwise actuates the second actuation mechanism 30, which imparts rotational force to or otherwise actuates the belt/chain drive system 31 or the like, which imparts rotational force to or otherwise drives the gear, thereby extending/retracting the bridge member 26 from/to the elongated stem 16. Alternatively, a powered mechanism (not illustrated) may be attached to and actuate the belt/chain drive system 31 or the like responsive to the surgeon's command. The belt/chain drive system 31 or the like may be either substantially disposed within or external to the elongated stem 16 and, in the later case, it may be disposed within a separate housing (not illustrated).

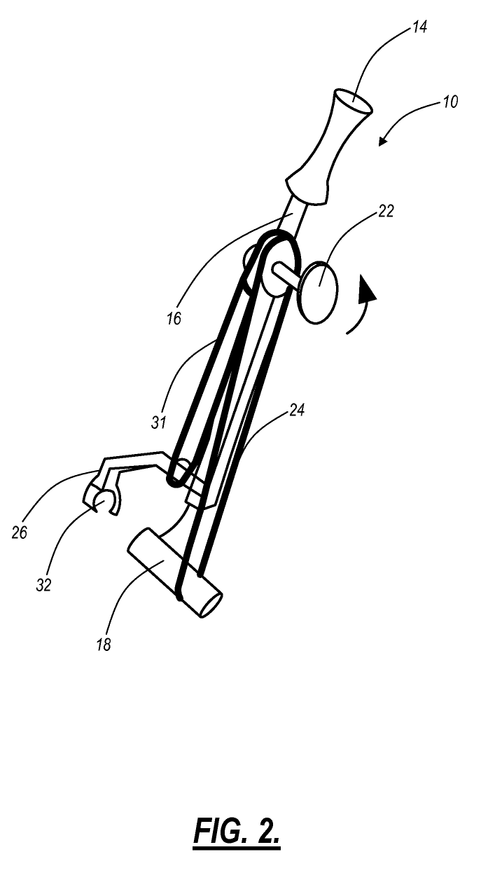

Referring to FIG. 2, again, a first actuation mechanism 22 is located on the elongated stem 16 for driving the facet bolt 12 (FIG. 1) into and through the spinal facet joint, preferably but not necessarily by imparting a rotational force to the facet bolt 12. In this exemplary embodiment, the first actuation mechanism 22 includes a knob that is rotatably connected to the elongated stem 16. A belt/chain drive system 24 or the like is rotatably connected to the first actuation mechanism 22, as well as to a gear 52 (FIG. 3) associated with the housing 18 and internally threaded mechanism 20 (FIG. 1). In operation, the surgeon rotates or otherwise actuates the first actuation mechanism 22, which imparts rotational force to or otherwise actuates the belt/chain drive system 24 or the like, which imparts rotational force to or otherwise drives the gear 52, thereby driving the facet bolt 12 into and through the spinal facet joint. Alternatively, a powered mechanism (not illustrated) may be attached to and actuate the belt/chain drive system 24 or the like responsive to the surgeon's command. The belt/chain drive system 24 or the like may be either substantially disposed within or external to the elongated stem 16 and, in the later case, it may be disposed within a separate housing (not illustrated).

Prior to driving the facet bolt 12 into and through the spinal facet joint, the unilateral facet bolt inserter 10 is positioned with respect to and secured to the spinal facet joint. This is accomplished using a bridge member 26 that is movably connected to the elongated stem 16 via an extensible threaded mechanism 28 (FIG. 1). The bridge member 26 is configured to receive the end of the facet bolt 12 after insertion through the spinal facet joint. Accordingly, either one or both of the housing 18 and the bridge member 26 may selectively retain a washer 32 and 33 (FIG. 1) through which the facet bolt 12 selectively passes, thereby retaining the facet bolt 12 in the spinal facet joint when the unilateral facet bolt inserter 10 is removed. One or both of these washers 32 and 33 may have any suitable bone engaging structures, such as friction surfaces, teeth, etc.

The bridge member 26 is configured to selectively move towards the facet bolt 12 prior to or during insertion to lock the unilateral facet bolt inserter 10 onto the spinal facet joint. A second actuation mechanism 30 (FIG. 1) is located on the elongated stem 16 for this purpose. In this exemplary embodiment, the second actuation mechanism 30 includes a knob that is rotatably connected to the elongated stem 16. A belt/chain drive system 31 or the like is rotatably connected to the second actuation mechanism 30, as well as to a gear (not illustrated) associated with the bridge member 26. In operation, the surgeon rotates or otherwise actuates the second actuation mechanism 30, which imparts rotational force to or otherwise actuates the belt/chain drive system 31 or the like, which imparts rotational force to or otherwise drives the gear, thereby extending/retracting the bridge member 26 from/to the elongated stem 16. Alternatively, a powered mechanism (not illustrated) may be attached to and actuate the belt/chain drive system 31 or the like responsive to the surgeon's command. The belt/chain drive system 31 or the like may be either substantially disposed within or external to the elongated stem 16 and, in the later case, it may be disposed within a separate housing (not illustrated).

Referring to FIG. 3, the housing 18 includes the internally threaded mechanism 20 described above that is coupled to the gear 52 also described above. Upon rotation, the gear 52 drives an associated capture rod 56 and 58 that is selectively coupled to the facet bolt 12 via a hex fitting or the like. The internally threaded mechanism 20 and capture rod are allowed motion relative to the housing 18 via a bearing assembly 54 disposed within the housing 18. It will be readily apparent to those of ordinary skill in the art that other suitable mechanisms for driving the facet bolt 12 may be utilized equally, and that the mechanism illustrated and described herein is exemplary only.

Referring now to FIGS. 1 and 3, the facet bolt 12 includes a fastener having an elongated shaft and a head. The fastener engages the first washer 32 and second washer 33 and a locking member (not illustrated). The head may have a convex surface for use in engaging the first washer 32 as is described in greater detail herein. Threads are provided on the elongated shaft for the purpose of engaging the locking member. Other engagement structures may also be used. A pointed end is provided for piercing bone during the implantation process. Grooves or flutes provide space for bone chips to disperse during insertion into the locking member and to carry bone debris away from the pointed end as it is progressing through the bone. A suitable structure, such as a hex opening or the like, is provided for engagement of the fastener to apply compressive and/or rotational forces during the implantation process. Depressions or other suitable structures may be provided to permit a fastening device to grip the head.

The first and second washers 32 and 33 may have any suitable structures, such as serrations, for engaging the bone surface. Alternative structures are also possible. The first and/or second washers 32 and 33 are pivotal with respect to the locking member. The locking member may have any suitable structures, such as apertures. Cooperating engagement structures, such as a clip, may be provided for extending through apertures in the first and/or second washers 32 and 33 and engaging the apertures, such that the first and/or second washers 32 and 33 engage the locking member. A convex surface on the locking member may cooperate with a similar concave surface on the first and/or second washers 32 and 33. The clip may be positioned in a suitable retaining groove on the first and/or second washers 32 and 33. An aperture and slotted L-shaped groove may be provided to engage corresponding protrusions on the lower end portion to secure the locking member to the lower end portion of the fastening device.

The first and/or second washers 32 and 33 may have a concave surface for cooperating with the convex surface of the head of the fastener. An elongated opening, which may be in the form of a tapered slot, permits the pivoting of the first and/or second washers 32 and 33 relative to the fastener. The first and/or second washers 32 and 33 may have any structure for allowing the washer to be engaged by the fastening device. This structure may be a circumferential groove that may be engaged by a cooperating flange structure on the fastening device. This allows the first and/or second washers 32 and 33 to be advanced toward the surface of the bone. Additional structures, such as depressions, may be provided for engagement by the fastening device to permit rotation of the first and/or second washers 32 and 33. This assists in properly positioning the first and/or second washers 32 and 33. The first and/or second washers 32 and 33 may have an angled contact surface, which permits the first and/or second washers 32 and 33 to cooperate against bone surfaces, such as facets, which present significant angles. Additional structures may be provided to promote engagement. These structures may include serrations on the contact surface(s). The opening may expand wider in a fluted manner toward the contact surface(s) to permit greater pivoting.

The manner of engagement between the fastener, the first and/or second washers 32 and 33, and the locking member is flexible. The first and/or second washers 32 and 33 may tilt approximately 45° from a transverse section through the screw axis. More or less is possible, depending on the bone system that is being fused.

Any suitable surgically implantable material, metal, plastic, or composite, may be used to form any/all of the components illustrated and described herein, and any appropriate dimensions may be utilized.

Although the present invention is illustrated and described herein with reference to preferred embodiments and specific examples thereof, it will be readily apparent to those of ordinary skill in the art that other embodiments and examples may perform similar functions and/or achieve like results. For example, although the unilateral facet bolt inserter has been illustrated and described herein primarily in relation to spinal surgical procedures and facet joints, it may find equal applicability to other surgical procedures and joints or bony structures. All such equivalent embodiments and examples are within the spirit and scope of the present invention, are contemplated thereby, and are intended to be covered by the following claims.

What is claimed is:

1. A surgical device for securing two bony structures together, comprising:
a handle;
an elongated stem coupled to the handle;
a housing coupled to the elongated stem;
a bolt driving assembly disposed at least partially within the housing, wherein the bolt driving assembly is operable for selectively retaining a bolt and driving it through the two bony structures; and
a bridge member coupled to the elongated stem, wherein the bridge member is selectively extensible from/retractable to the elongated stem and is operable for selectively retaining a first washer that is configured and positioned to selectively receive the bolt after it is driven through the two bony structures.

2. The surgical device of claim 1, wherein the housing is articulatable with respect to the elongated stem.

3. The surgical device of claim 1, further comprising a first actuation mechanism coupled to the elongated stem, wherein the first actuation mechanism is operable for actuating the bolt driving assembly.

4. The surgical device of claim 3, wherein the first actuation mechanism is operable for imparting rotational motion to the bolt driving assembly, whereby the bolt is rotated and translated through the two bony structures.

5. The surgical device of claim 1, further comprising a second actuation mechanism coupled to the elongated stem, wherein the second actuation mechanism is operable for actuating the bridge member.

6. The surgical device of claim 5, wherein the second actuation mechanism is operable for imparting rotational motion to the bridge member, whereby the bridge member is translated to the two bony structures.

7. The surgical device of claim 1, wherein one or more of the housing and the bolt driving assembly are operable for selectively retaining a second washer that is configured and positioned to selectively receive the bolt before it is driven through the two bony structures.

8. The surgical device of claim 1, wherein the two bony structures comprise the superior and inferior facets of a facet joint of a spine of a patient.

9. A surgical method for securing two bony structures together, comprising:
providing a handle;
providing an elongated stem coupled to the handle;
providing a housing coupled to the elongated stem;
providing a bolt driving assembly disposed at least partially within the housing, wherein the bolt driving assembly is operable for selectively retaining a bolt and driving it through the two bony structures; and
providing a bridge member coupled to the elongated stem, wherein the bridge member is selectively extensible from/retractable to the elongated stem and is operable for selectively retaining a first washer that is configured and positioned to selectively receive the bolt after it is driven through the two bony structures.

10. The surgical method of claim 9, wherein the housing is articulatable with respect to the elongated stem.

11. The surgical method of claim 9, further comprising providing a first actuation mechanism coupled to the elongated stem, wherein the first actuation mechanism is operable for actuating the bolt driving assembly.

12. The surgical method of claim 11, wherein the first actuation mechanism is operable for imparting rotational motion to the bolt driving assembly, whereby the bolt is rotated and translated through the two bony structures.

13. The surgical method of claim 9, further comprising providing a second actuation mechanism coupled to the elongated stem, wherein the second actuation mechanism is operable for actuating the bridge member.

14. The surgical method of claim 13, wherein the second actuation mechanism is operable for imparting rotational motion to the bridge member, whereby the bridge member is translated to the two bony structures.

15. The surgical method of claim 9, wherein one or more of the housing and the bolt driving assembly are operable for selectively retaining a second washer that is configured and positioned to selectively receive the bolt before it is driven through the two bony structures.

16. The surgical method of claim 9, wherein the two bony structures comprise the superior and inferior facets of a facet joint of a spine of a patient.

17. A surgical method for securing two bony structures together, comprising:
disposing a bolt driving assembly and a bridge member on opposing sides of a joint comprising the two bony structures,
wherein the bolt driving assembly is operable for selectively retaining a bolt and driving it through the two bony structures, and
wherein the bridge member is selectively extensible from/retractable to the joint and is operable for selectively retaining a first washer that is configured and positioned to selectively receive the bolt after it is driven through the two bony structures;
securing the two bony structures between the bolt driving assembly and the bridge member; and
driving the bolt through the two bony structures and the first washer.

18. The surgical method of claim 17, wherein the bolt driving assembly is operable for selectively retaining a second washer that is configured and positioned to selectively receive the bolt before it is driven through the two bony structures.

19. The surgical method of claim 17, wherein the two bony structures comprise the superior and inferior facets of a facet joint of a spine of a patient.
